(19) United States
(12) Patent Application Publication
(10) Pub. No.: US 2008/0271205 A1
(43) Pub. Date: Oct. 30, 2008

(54) Method for producing steviol synthetase gene and steviol

(75) Inventors: Shinjiro Yamaguchi, Kanagawa (JP); Takahito Nomura, Kanagawa (JP); Hiroshi Magome, Kanagawa (JP); Yuji Kamiya, Kanagawa (JP)

Correspondence Address: BIRCH STEWART KOLASCH & BIRCH, PO BOX 747, FALLS CHURCH, VA, 22040-0747, US

(73) Assignee: RIKEN

(21) Appl. No.: 12/078,016

(22) Filed: Mar. 26, 2008

(30) Foreign Application Priority Data
Mar. 27, 2007 (JP) 2007-082193

(51) Int. Cl. C12N 15/82; C12N 15/11; C12N 15/00; C12N 5/04; A01H 5/00; C12P 21/04

(52) U.S. Cl. 800/278; 536/23.2; 435/320.1; 435/419; 800/298; 435/69.1

(57) ABSTRACT

To identify the steviol synthetase gene for research and development of metabolic engineering, for example, for increasing a stevioside producing ability. It was successfully found that CYP714A2 derived from Arabidopsis thaliana is surprisingly steviol synthetase. Furthermore, a system in which a large amount of steviol can be biosynthesized was developed by overexpressing this steviol synthetase gene. The steviol synthetase gene is, for example, a polynucleotide encoding a protein which includes the amino acid sequence of SEQ ID NO: 2.

### BACKGROUND OF THE INVENTION

1. Field of the Invention

The present invention relates to a steviol synthetase gene encoding an enzyme which has an activity of synthesizing steviol by hydroxylation of the 13th carbon of ent-kaurenoic acid.

2. Background Art

Among cytochrome P450 enzymes, CYP714A2 derived from Arabidopsis thaliana is known to belong to the same family as CYP714D1 of rice plant. CYP714D1 of rice plant has a function of catalyzing epoxidation of gibberellin at the 16th (17th) carbon (Non-patent Document 1). It is therefore predicted that CYP714A2 derived from Arabidopsis thaliana also has the same function, but an actual function thereof in the organism remains unknown.

Meanwhile, steviol is an aglycon of stevioside, which is a natural sweetener produced by stevia (Stevia rebaudiana). Studies of stevioside biosynthetic enzymes including approaches such as collection of a large amount of expressed sequence tags (ESTs) have been actively conducted (Non-patent Document 2), and more than one enzyme involved in glycosylation of steviol (glucosyltransferase) has been identified. However, no steviol synthetase gene has been identified, and it has been very difficult to increase a stevioside producing ability in metabolic engineering for this reason.

Furthermore, most of enzymes involved in biosynthesis and metabolism of gibberellin, which is a plant growth hormone, have been identified, but a gene encoding an enzyme for C-13 hydroxylation in gibberellin has not been identified.

[Non-patent Document 1] Zhu et al., ELONGATED UPPERMOST INTERNODE encodes a cytochrome P450 monooxygenase that epoxidizes gibberellins in a novel deactivation reaction in rice. Plant Cell, 18: 442-456 (2006)
[Non-patent Document 2] Richman A, Swanson A, Humphrey T, Chapman R, McGarvey B, Pocs R, Brandle J. Functional genomics uncovers three glucosyltransferases involved in the synthesis of the major sweet glucosides of Stevia rebaudiana. Plant J. 41: 56-67 (2005)

### SUMMARY OF THE INVENTION

Thus, no steviol synthetase gene has been identified, and it has been desired that this gene will be identified for research and development of metabolic engineering to increase a stevioside producing ability, for example. However, there is a problem that no finding about the steviol synthetase gene has been obtained to date.

Accordingly, the inventors of the present invention conducted various researches to solve the above-mentioned problem. As a result, they successfully found that CYP714A2 derived from Arabidopsis thaliana belonging to the same family as CYP714D1 rice plant is surprisingly a steviol synthetase. Furthermore, the inventors of the present invention developed a system in which a large amount of steviol can be biosynthesized by overexpressing this steviol synthetase gene, and found interesting phenotypes. Thus, the present invention was accomplished.

The present invention includes the following.

(1) a steviol synthetase gene comprising any of the following polynucleotides (a) to (c):
(a) a polynucleotide encoding a protein which comprises the amino acid sequence of SEQ ID NO: 2;
(b) a polynucleotide encoding a protein which comprises an amino acid sequence of SEQ ID NO: 2 with deletion, substitution, addition, or insertion of one or more amino acids and has a function of hydroxylating the 13th carbon of ent-kaurenoic acid; or
(c) a polynucleotide encoding a protein which comprises an amino acid sequence having homology of 70% or more with the amino acid sequence of SEQ ID NO: 2 and has a function of hydroxylating the 13th carbon of ent-kaurenoic acid.

Furthermore, the steviol synthetase gene of the present invention is not particularly limited, but is preferably derived from a plant selected from the group consisting of, for example, Arabidopsis thaliana, rice plant, Populus nigra, Rubus suavissimus, and stevia. Furthermore, the steviol synthetase gene of the present invention may be provided as an expression vector, or a transformed cell or a transformed plant into which the gene is functionally incorporated.

Furthermore, the method for producing steviol of the present invention comprises the step of extracting steviol from a transformant into which the steviol synthetase gene of the present invention is incorporated so as to be overexpressed. At this time, it is preferable to add ent-kaurenoic acid, which is a substrate of steviol synthetase encoded by the steviol synthetase gene of the present invention. The method for producing steviol of the present invention can be applied to a transformed plant into which the steviol synthetase gene is incorporated or a transformed gibberellin producing fungus.

Furthermore, the method for changing a ratio of gibberellin A₁ and gibberellin A₄ of the present invention comprises the step of overexpressing the steviol synthetase gene of the present invention in a target cell.

Since the steviol synthetase gene of the present invention encodes an enzyme having an activity of hydroxylating the 13th carbon of ent-kaurenoic acid, it can be utilized in a steviol synthesis system. For example, a large amount of steviol can be produced in an organism such as a plant by overexpressing the steviol synthetase gene of the present invention in this organism.

Furthermore, according to the present invention, a method for regulating plant morphology can be provided in which semidwarfism is exhibited by overexpressing the steviol synthetase gene in a plant, and semidwarfism is recovered by exogenously dosing active gibberellin A₄. Furthermore, according to the present invention, a ratio of gibberellin A₁ and gibberellin A₄ in a cell can be changed by overexpressing the steviol synthetase gene in the cell.

### BRIEF DESCRIPTION OF THE DRAWINGS

FIG. 1 is a schematic characteristic diagram of a hydroxylation reaction by the steviol synthetase of present invention.

FIG. 2 is a schematic characteristic diagram of a system which produces various glycosides by utilizing the steviol synthetase gene of the present invention without steviol synthesis being a rate-determining step.

FIG. 3A is a photograph of wild-type Arabidopsis thaliana. FIG. 3B is a photograph of steviol synthetase gene overexpressing Arabidopsis thaliana.

FIG. 4 is a photograph of steviol synthetase gene overexpressing Arabidopsis thaliana, wild-type Arabidopsis thaliana, and GA synthesis failure mutants.

FIG. 5 is a characteristic diagram showing results of comparison of the rosette radii of steviol synthetase gene overexpressing Arabidopsis thaliana, wild-type Arabidopsis thaliana, and GA synthesis failure mutants.

### DETAILED DESCRIPTION OF THE PREFERRED EMBODIMENTS

Hereafter, the present invention will be described in detail with reference to the accompanying drawings.

### 1. Novel Steviol Synthetase Gene

The steviol synthetase gene of the present invention is a gene encoding an enzyme having an activity of hydroxylating the 13th carbon of ent-kaurenoic acid (steviol synthetase). This steviol synthetase can be isolated from plants and fungi known to produce various glycosides with steviol as an aglycon. Examples thereof include the steviol synthetase gene derived from Arabidopsis thaliana. The nucleotide sequence of the steviol synthetase gene derived from Arabidopsis thaliana is shown as SEQ ID NO: 1. The amino acid sequence of the steviol synthetase derived from Arabidopsis thaliana is shown as SEQ ID NO: 2.

The nucleotide sequence of SEQ ID NO: 1 is known to encode cytochrome P450 enzyme CYP714A2 of Arabidopsis thaliana, but it has been unknown that this CYP714A2 has an activity of hydroxylating the 13th carbon of ent-kaurenoic acid. No enzyme having an activity of hydroxylating the 13th carbon of ent-kaurenoic acid has been identified, and it is a completely new finding that the protein having the amino acid sequence of SEQ ID NO: 2 is steviol synthetase. The hydroxylation reaction of the 13th carbon of ent-kaurenoic acid is represented by the following formula.

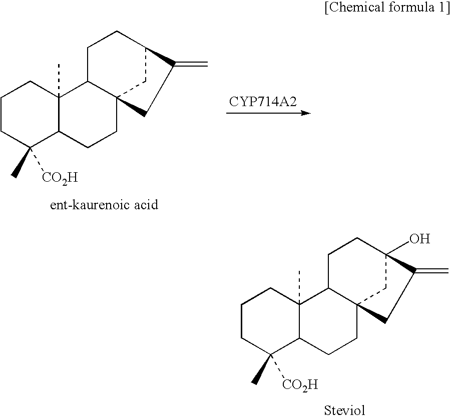

Furthermore, the steviol synthetase gene of the present invention is not limited to the gene derived from Arabidopsis thaliana, and genes may be derived from plants and fungi in which steviol or a glycoside thereof is accumulated.

Furthermore, the steviol synthetase gene of the present invention may be a gene comprising a polynucleotide encoding a protein which has an amino acid sequence of SEQ ID NO: 2 including deletion, substitution, addition, or deletion of one or more amino acids and a function of hydroxylating the 13th carbon of ent-kaurenoic acid. The expression “one or more amino acids” here means, for example, one to 20 amino acids, preferably one to 10 amino acids, more preferably one to five amino acids.

Furthermore, the steviol synthetase gene of the present invention may comprise a polynucleotide encoding a protein which has an amino acid sequence having homology of 70% or more, preferably 80% or more, more preferably 90% or more with the amino acid sequence of SEQ ID NO: 2 and a function of hydroxylating the 13th carbon of ent-kaurenoic acid. Here, the homology of amino acid sequence can be determined using the BLAST algorithm (Proc. Natl. Acad. Sci. USA 87: 2264-2268, 1990, Proc Natl Acad Sci USA 90: 5873, 1993). Homology between amino acid sequences can be calculated using a program called BLASTX based on the BLAST algorithm (Altschul S F, et al: J Mol Biol 215: 403, 1990), and default values can be used for parameters.

Furthermore, the steviol synthetase gene of the present invention may be a polynucleotide hybridizable with a probe comprising the whole or a part of the nucleotide sequence of SEQ ID NO: 1 or a strand complementary thereto under a stringent condition and encoding a protein having a function of hydroxylating the 13th carbon of ent-kaurenoic acid. As a probe hybridizable under a stringent condition, a polynucleotide obtained by selecting one or more from at least 20, preferably at least 30, for example, 40, 60, or 100 arbitrary continuous sequences in the nucleotide sequence of SEQ ID NO: 1 can be used. The “stringent condition” here is a condition under which a signal of a specific hybrid is clearly distinguished from a signal of a non-specific hybrid. The stringent condition is exemplified by a condition under which hybridization is performed using 5×SSC, 1.0% (W/V) nucleic acid hybridization blocking reagent (Boehringer-Mannheim), 0.1% (W/V) N-lauroylsarcosine, and 0.02% (W/V) SDS (approximately 8 to 16 hours), followed by two washes using 0.1×SSC and 0.1% (W/V) SDS for 15 minutes. Furthermore, examples of temperatures for hybridization and wash include 67° C. or higher. Hybridization can be performed according to the methods described in “Molecular Cloning: A Laboratory Manual, 2nd Ed., Cold Spring Harbor Laboratory, Cold Spring Harbor, N.Y. (1989)” or “Current Protocols in Molecular Biology, Supplement 1-38, John Wiley & Sons (1987-1997).”

Meanwhile, a polynucleotide encoding an amino acid sequence of SEQ ID NO: 2 including deletion, substitution, addition, or insertion of one or more amino acids, a polynucleotide encoding an amino acid sequence having homology of 70% or more, preferably 80% or more, more preferably 90% or more with the amino acid sequence of SEQ ID NO: 2, and a polynucleotide hybridizable with a probe consisting of the whole or a part of the nucleotide sequence of SEQ ID NO: 1 or a strand complementary thereto under a stringent condition can be prepared by an arbitrary method known to those skilled in the art such as chemical synthesis, genetic engineering technique, or mutation induction based on information on the nucleotide sequence of SEQ ID NO: 1 and the amino acid sequence of SEQ ID NO: 2.

For example, a polynucleotide encoding an amino acid sequence of SEQ ID NO: 2 including deletion, substitution, addition, or insertion of one or more amino acids can be prepared by using a method comprising bringing a polynucleotide having the nucleotide sequence of SEQ ID NO: 1 into contact with an agent as a mutagen, a method comprising irradiating an ultraviolet ray, genetic engineering techniques, and the like. Site specific mutation induction, one of genetic engineering techniques, is useful because a specific mutation can be introduced into a specific site with this technique and can be performed according to the methods described in “Molecular Cloning: A Laboratory Manual, 2nd Ed., Cold Spring Harbor Laboratory, Cold Spring Harbor, N.Y. (1989)” and “Current Protocols in Molecular Biology, Supplement 1-38, John Wiley & Sons (1987-1997).”

Whether a polynucleotide having a predetermined nucleotide sequence is the steviol synthetase gene can be verified as follows. Specifically, a transformant into which a gene comprising a polynucleotide to be examined is functionally introduced is cultured in a medium containing ent-kaurenoic acid as a substrate. Then, components contained in an extract from the medium are loaded on a gas chromatography-mass spectrometry apparatus to confirm that steviol has been synthesized as a metabolite of ent-kaurenoic acid. Detection of steviol demonstrates that the gene comprising the polynucleotide is a steviol synthetase gene.

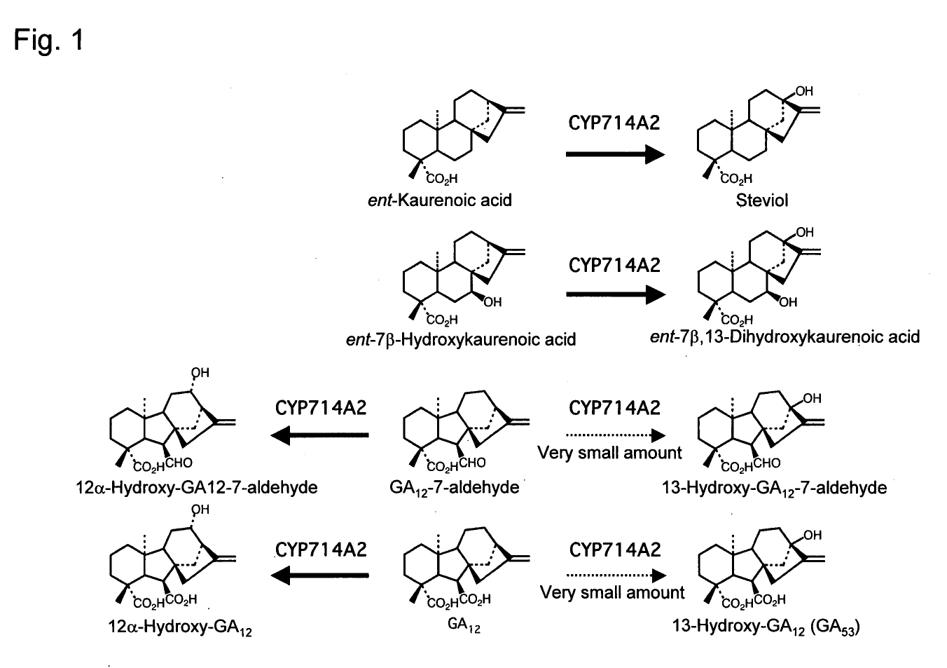

Meanwhile, as described above, the steviol synthetase gene of the present invention encodes steviol synthetase having an activity of hydroxylating the 13th carbon of ent-kaurenoic acid, but activities of this steviol synthetase are not limited to the activity of hydroxylating the 13th carbon of ent-kaurenoic acid. Specifically, this steviol synthetase also has an activity of hydroxylating the 13th carbon of ent-7β-hydroxykaurenoic acid. Furthermore, this steviol synthetase also has an activity of hydroxylating the 12th carbon of gibberellin A₁₂-7-aldehyde, but hydroxylates the 13th carbon of gibberellin A₁₂-7-aldehyde only to a small extent. Furthermore, this steviol synthetase gene also has an activity of hydroxylating the 12th carbon of gibberellin A₁₂, but hydroxylates the 13th carbon of gibberellin A₁₂ only to a small extent. These hydroxylation activities of steviol synthetase are schematically shown in FIG. 1.

### 2. Expression Vector

The steviol synthetase gene explained in the above 1. can be used and stored by inserting it into a suitable vector. Types of the vector into which the gene is inserted are not particularly limited, and the vector may be, for example, an autonomously replicating vector or a vector introduced into the genome of a host cell and replicated together with the chromosome into which the vector is incorporated. It is particularly preferable to use a vector into which the above-described steviol synthetase gene can be functionally incorporated. Specifically, it is preferable to prepare a vector as an expression vector harboring the above-described steviol synthetase gene. In the expression vector, components required for transcription (for example, promoter, etc.) are functionally ligated to the steviol synthetase gene. A promoter is a DNA sequence that exhibits a transcription activity in a host cell and can be suitably selected depending on the type of the host cell.

Examples of promoters that can function in bacterial cells include promoters for the Bacillus stearothermophilus maltogenic amylase gene, the Bacillus licheniformis alpha-amylase gene, the Bacillus amyloliquefaciens BAN amylase gene, the Bacillus Subtilis alkaline protease gene, the Bacillus pumilus xylosidase gene, the PR or PL promoter of phage lambda, lac, trp, or tac promoter of Escherichia coli, and so forth.

Examples of promoters that can function in mammal cells include the SV40 promoter, the metallothionein gene (MT-1) promoter, the adenovirus-2 major late promoter, and so forth. Examples of promoters that can function in insect cells include the polyhedrin promoter, the P10 promoter, the Autographa californica polyhedrosis basic protein promoter, the baculovirus immediate early gene 1 promoter, or the baculovirus 39K delayed early gene promoter, and so forth. Examples of promoters that can function in yeast host cells include promoters derived from yeast glycolysis system genes, alcohol dehydrogenase gene promoters, the TPI1 promoter, the ADH2-4-c promoter, and so forth. Examples of promoters that can function in filamentous fungi cells include the ADH3 promoter, the tpiA promoter, and so forth.

The expression vector may further contain a selection marker. Examples of selection markers include genes represented by dihydrofolic acid reductase (DHFR), the Schizosaccharomyces pombe TPI gene, and so forth, whose complement is deficient in the host cell, and drug-resistant genes such as ampicillin, kanamycin, tetracycline, chloramphenicol, neomycin, and hygromycin.

Furthermore, when an expression vector is constructed to overexpress the above-described steviol synthetase gene in a plant as a part of the purpose, any vector that can express the steviol synthetase gene in a plant can be used. Specific examples thereof include vectors which can be incorporated into the genome of a host plant when a part of DNA of a vector derived from the Ti plasmid of Agrobacterium tumefaciens is introduced into a plant cell, for example, pKYLX6, pKYLX7, pBI101, pBH2113, pBI121, and so forth derived from the Ti plasmid (Clontech Laboratories, Inc.).

As promoters which can function in plants, promoters derived from the target plant or those derived from other types of plants can be used so long as they function in the target plant. Furthermore, as required, externally inducible promoters and tissue specific promoters can also be used. The CaMV35 promoter, the NOS promoter and the octopine synthase promoter, promoters which are tissue-nonspecific but exhibit a potent expression inducing property (Fromm et al. [1989] Plant Cell 1: 977), can also be used. Furthermore, the rbcS promoter and the cab promoter, which induce strong expression in green leaves, can also be used (Chory et al. [1991], Plant Cell, 3, 445-459). Estradiol inducing promoters (Plant Cell 2000; 12: 65-80), pUAS-Gal4 glucocorticoid inducing promoters (Plant J. 11, 605-612), and the like can also be used. Furthermore, specific examples of promoters include promoters derived from T-DNA of Agrobacterium tumefaciens, the Smas promoter, the cinnamon alcohol dehydrogenase promoter, the ribulose diphosphate carboxylase oxygenase (Rubisco) promoter, the GRP1-8 promoter, promoters/enhancers of plant-derived actins, histones, and the like, and other transcription initiation regions of known various plant genes fall within the scope of the present invention.

Furthermore, to efficiently express the steviol synthetase gene, the poly(A)+ sequence may be added to the 3′ end of the coding region of the gene. Poly(A)+ sequences derived from various plant genes or T-DNAs can be used, but they are not limited to these examples. Furthermore, to express this gene at a high level, other useful sequences, such as, for example, intron sequences and the 5′ untranslated region sequence of a specific gene can be included in the expression vector.

Furthermore, various antibiotic resistance genes and other marker genes can be included in the expression vector as selection marker genes. Examples of marker genes include the anti-spectinomycin gene, the streptomycin resistance gene (the streptomycin phosphotransferase [SPT] gene), the neomycin phosphotransferase (NPTII) gene for kanamycin or geneticin resistance, the hygromycin phosphotransferase (HPT) gene for hygromycin resistance, genes for resistance to a herbicide inhibiting acetolactate synthetase (ALS), genes for resistance to a herbicide inhibiting glutamine synthetase (for example, the bar gene), the β-glucuronidase gene, the luciferase gene, and so forth.

### 3. Transformant

A transformant can be prepared by introducing the expression vector explained in the above 2. into a host cell. The host cell may be an arbitrary cell so long as the steviol synthetase gene incorporated in the expression vector can be expressed, and may be any of bacterial, yeast, fungal, animal, insect and/or plant cells.

Examples of bacteria include Gram-positive bacteria such as Bacillus and Streptomyces and Gram-negative bacteria such as Escherichia coli. These bacteria can be transformed by a protoplast method or by a known method using competent cells. Examples of mammalian cells include the HEK293 cell, the HeLa cell, the COS cell, the BHK cell, the CHL cell, the CHO cell, and so forth. Methods for transforming a mammalian cell and expressing a DNA sequence introduced into the cell are also known, and electroporation methods, calcium phosphate methods, lipofection methods, and the like can be used, for example. Examples of yeasts include cells belonging to the genera Saccharomyces and Schizosaccharomyces, such as, for example, Saccharomyces cerevisiae and Saccharomyces kluyveri. Examples of methods for introducing a recombinant vector into a yeast host include electroporation methods, spheroblast methods, lithium acetate methods, and so forth.

Furthermore, fungi are not particularly limited, but it is preferable to use fingi known as gibberellin producing fungi. Examples of gibberellin producing fungi include Gibberella fujikuroi, Phaeosphaeria sp. L487, and so forth. Since these gibberellin producing fungi are considered to accumulate a large amount of ent-kaurenoic acid used as a substrate of steviol synthetase by metabolism, it is expected that they can synthesize a large amount of steviol utilizing the accumulated ent-kaurenoic acid by overexpressing the steviol synthetase gene.

Furthermore, a plant can be transformed by applying, for example, particle gun methods, electroporation methods, polyethylene glycol (PEG) methods, calcium phosphate methods, DEAE dextran methods, microinjection methods, lipofection methods, and transfection methods mediated by microorganisms such as Agrobacterium, using the expression vector explained in the above 2. For plant cells, particle gun methods, electroporation methods, polyethylene glycol (PEG) methods, and Agrobacterium methods are preferably used, and Agrobacterium methods are particularly preferable (Bechtold N. & Pelletier G., Methods Mol. Biol. 82, pp. 259-266, 1998).

A plant to be transformed means any of the whole plant, a plant organ (for example, leaf, petal, stem, root, seed, etc.), a plant tissue (for example, epidermis, phloem, parenchyma, xylem, vascular bundle, palisade tissue, spongy parenchyma, etc.), or a plant cultured cell (for example, callus). Plants used for transformation are not limited, but the following plants are possible, for example.

Solanaceae family: eggplant (Solanum melongena L.), tomato (Lycopersicon esculentum Mill), green pepper (Capsicum annuum L. var. angulosum Mill.), red pepper (Capsicum annuum L.), tobacco (Nicotiana tabacum L.)
Brassicaceae family: thale cress (Arabidopsis thaliana), rape (Brassica campestris L.), Chinese cabbage (Brassica pekinensis Rupr.), cabbage (Brassica oleracea L. var. capitata L.), radish (Raphanus sativus L.), rapeseed (Brassica campestris L., B. napus L.)
Gramineae family: maize (Zea mays), rice plant (Oryza sativa), wheat (Triticum aestivum L.), barley (Hordeum vulgare L.)
Leguminosae family: soybean (Glycine max), adzuki bean (Vigna angularis Willd.), bush bean (Phaseolus vulgaris L.), broad bean (Vicia faba L.)
Cucurbitaceae family: cucumber (Cucumis sativus L.), melon (Cucumis melo L.), watermelon (Citrullus vulgaris Schrad.), pumpkin (C. moschata Duch., C. maxima Duch.)
Convolvulaceae family: sweet potato (Ipomoea batatas)
Liliaceae family: spring onion (Allium fistulosum L.), onion (Allium cepa L.), nira (Allium tuberosum Rottl.), garlic (Allium sativum L.), asparagus (Asparagus officinalis L.)
Labiatae family: perilla (Perilla frutescens Britt. var. crispa)
Aster family: chrysanthemum (Chrysanthemum morifolium), garland chrysanthemum (Chrysanthemum coronarium L.), lettuce (Lactuca sativa L. var. capitata L.)
Rosaceae family: rose (Rose hybrida Hort.), strawberry (Fragaria x ananassa Duch.)
Rutaceae family: mandarin orange (Citras unshiu), Japanese pepper (Zanthoxylum piperitum DC.)
Myrtaceae family: eucalyptus (Eucalyptus globulus Labill.)
Salicaceae family: black poplar (Populus nigra L. var. italica Koehne)
Chenopodiaceae family: spinach (Spinacia oleracea L.), beet (Beta vulgaris L.)
Gentianaceae family: gentian (Gentiana scabra Bunge var. buergeri Maxim.)
Caryophyllaceae family: carnation (Dianthus caryophyllus L.)

In particular, plants known to biosynthesize various glycosides with steviol as an aglycon are preferably used as plants to be transformed. Examples of such plants include stevia (Stevia rebaudiana), Tencha (Rubus suavissimus), and so forth. Examples of plants to be transformed further include black poplar (Populus nigra L. var. italica Koehne) and the like, which are studied for use as a biomass.

Tumor tissues, shoots, capillary roots, and the like obtained as a result of transformation can be used for cell culture, tissue culture, or organ culture as they are and can be regenerated in a plant body (transgenic plant) by dosing of appropriate concentrations of plant hormones (auxin, cytokinin, gibberellin, abscisic acid, ethylene, brassinolide, etc.) by known plant tissue culture methods.

Whether the steviol synthetase gene has been incorporated into a plant can be confirmed by the PCR method, the Southern hybridization method, the Northern hybridization method, or the like. For example, PCR is performed by preparing DNA from a transgenic plant and designing DNA-specific primers. PCR can be performed under the same conditions as conditions employed for amplification of cDNA fragment of the steviol synthetase gene inserted into the expression vector. Then, the amplification product is subjected to agarose gel electrophoresis, polyacrylamide gel electrophoresis, capillary electrophoresis, or the like and stained with ethidium bromide, a SYBR Green solution, or the like, then, transformation can be confirmed by detecting the amplification product as one band. Furthermore, the amplification product can also be detected by performing PCR using primers labeled with a fluorescent dye or the like beforehand. Furthermore, methods can be employed in which the amplification product is bound to a solid phase such as a microplate and confirmed by fluorescence or enzymatic reaction, or the like.

### 4. Method for Producing Steviol

Steviol can be biosynthesized by culturing or growing the transformant explained in the above 2. in the presence of ent-kaurenoic acid. Specifically, steviol synthetase expressed in a transformant hydroxylates the 13th carbon of ent-kaurenoic acid, and steviol can be produced. Here, ent-kaurenoic acid may be endogenous or exogenously dosed.

Furthermore, steviol biosynthesized in a transformant can be extracted by a usual method. For example, a cultured or grown transformant is extracted using an acetone solvent or an ethyl acetate/n-hexane (1:1) solvent, and steviol can be isolated and purified from the extract.

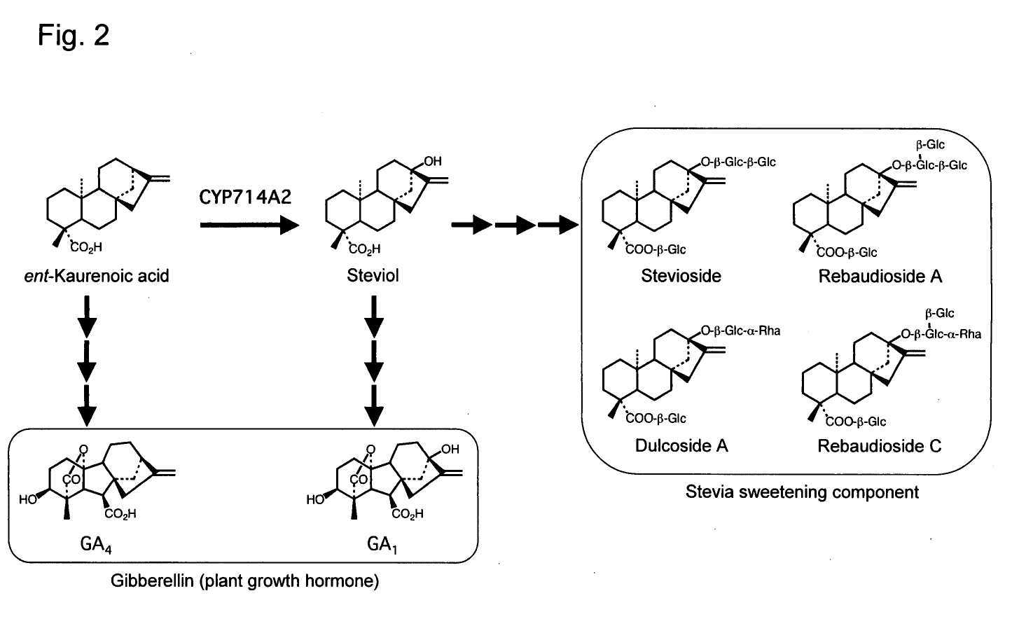

As described above, a steviol biosynthesis system can be developed by utilizing the steviol synthetase gene explained in the above 1, and steviol can be produced by biosynthesis. Conventionally, steviol synthesis has been a rate-determining step in a system in which various glycosides with steviol as an aglycon are produced by metabolic engineering. By utilizing the steviol synthetase gene explained in the above 1, however, a system for various glycosides can be developed without steviol synthesis being a rate-determining step (see FIG. 2).

Here, examples of glycosides with steviol as an aglycon include stevioside, rebaudioside A, rebaudioside B, rebaudioside C, rebaudioside D, rebaudioside E, rebaudioside F, dulcoside A, steviolmonoside, steviolbioside, rubusoside, and so forth. Thus, a system in which these various glycosides can be produced in an excellent yield can be developed utilizing the steviol synthetase gene explained in the above 1.

5. Phenotype Resulting from Overexpression of Steviol Synthetase Gene

As described above, when the steviol synthetase gene explained in the above 1. is overexpressed in a plant, synthesis of steviol is promoted. In addition, when the steviol synthetase gene explained in the above 1. is overexpressed in a plant, synthesis of gibberellin A₁ among gibberellins, plant growth hormones, is promoted. In wild-type plants, gibberellin A₄, which has a higher bioactivity than that of gibberellin A₁, is accumulated in a relatively large amount using ent-kaurenoic acid as a precursor. Furthermore, gibberellin A₁ has been conventionally thought to be synthesized by hydroxylation of gibberellin A₄.

However, since accumulation of gibberellin A₁ is increased when the steviol synthetase gene explained in the above 1. is overexpressed in a plant, it is highly probable that gibberellin A₁ is biosynthesized in a plant using steviol as a precursor. This is a finding against the above-described conventional prediction (see FIG. 2).

In other words, by overexpressing the steviol synthetase gene explained in the above 1. in a plant, the existing ratio of gibberellin A₄ and gibberellin A₁ in this plant can be changed as compared with that in a wild-type plant. Specifically, the value of (gibberellin A₁)/(gibberellin A₄) can be adjusted to increase as compared with a wild-type plant by overexpressing the steviol synthetase gene explained in the above 1. in a plant.

Furthermore, when the steviol synthetase gene explained in the above 1. in a plant is overexpressed, the plant body shows a characteristic phenotype such as semidwarfism. Specifically, a plant body overexpressing the steviol synthetase gene explained in the above 1. is significantly dwarfed as compared with a wild-type plant. Furthermore, the growth of a plant body showing semidwarfism recovers to a size comparable to that of a wild-type plant by exogenously dosing gibberellin A₄.

That is, a method for regulating a morphology of a plant comprising overexpressing the steviol synthetase gene explained in the above 1. in the plant to achieve semidwarfism, then recovering the growth by exogenously dosing gibberellin A₄ can be provided.

### EXAMPLES

The present invention will be explained more specifically with reference to the following examples. However, the technical scope of the present invention is not limited to these examples.

### Example 1

### Functional Testing of Novel Steviol Synthetase

The complete length cDNA of CYP714A2, a cytochrome P450 enzyme gene, was isolated from an immature pod of Arabidopsis thaliana by RT-PCR. The following primer set, the Expand High Fidelity PLUS PCR System (Roche), and the Pyrobest (Takara Bio Inc.) were used for RT-PCR. Then, the restriction enzyme sites were introduced by performing PCR using cDNA obtained by RT-PCR as a template and PCR primers 714A2-F1 (CGGGATCCATGGAGAGTTTGGTTGTTCATAC [SEQ ID NO: 3]: the BamHI restriction enzyme site was positioned immediately before the translation start codon [underlined]) and 714A2-R1 (GGGGTACCTCAAACAACCCTAATGACAACAC [SEQ ID NO: 4]: the KpnI restriction enzyme site was positioned immediately after the stop codon [underlined]). The product was digested with restriction enzymes BamHI and KpnI and ligated to the BamHI/KpnI site of the pYeDP60 vector. The pYeDP60 vector is a known vector which induces expression of a cytochrome P450 gene in the presence of galactose.

WAT11, a known yeast which coexpresses cytochrome P450 reduction enzyme 1 of Arabidopsis (Pompon D, Louerat B, Bronine A, Urban P, Yeast expression of animal and plant P450s in optimised redox environments. Methods Enzymol 272: 51-64 [1996]), was transformed with the obtained plasmid in the presence of galactose.

The obtained transformant was inoculated in 10 ml of SGI liquid medium (20 g of glucose, 6.7 g of yeast nitrogen base without amino acids, 1 g of bacto casamino acid, 40 mg of DL-tryptophan, 1 L of H₂O) and cultured at 30° C. for 24 hours with shaking (200 rpm). 1 ml of the culture broth was inoculated in 10 ml of SLI liquid medium (20 g of galactose, 6.7 g of yeast nitrogen base without amino acids, 1 g of bacto casamino acid, 40 mg of DL-tryptophan, 1 L of H₂O) and cultured at 28° C. with shaking until grown to a concentration of 4×10⁷ cells/ml. The grown transformant yeasts were diluted with a fresh SLI liquid medium to a concentration of 8×10⁶ cells/ml. 1 μg (dissolved in 1 μl of ethanol) each of ent-kaurenoic acid, ent-7β-hydroxykaurenoic acid, gibberellin A₁₂-7-aldehyde (hereinafter referred to as GA₁₂-7-aldehyde), and gibberellin A₁₂ (hereinafter referred to as GA₁₂) were added to 5 ml of the transformation medium and cultured at 28° C. with shaking until grown to a concentration of 6×10⁷ cells/ml. After culture, the transformant to which ent-kaurenoic acid, ent-7β-hydroxykaurenoic acid, and GA₁₂-7-aldehyde were added and the medium were extracted with ethyl acetate/n-hexane (1:1), and the extract was dried, then dissolved in 90% methanol, and allowed to pass through the Bond Elut C18 column (100 mg, Varian, Inc.). The transformant to which GA₁₂ was added and the medium were extracted with ethyl acetate, and the extract was dried, then dissolved in 80% methanol and allowed to pass through the Bond Elut C18 column. The eluate was dried, and then a methyl-TMSi derivative was obtained to analyze by GC-MS. The DB-1 column (0.25 mm×15 m; 0.25 μm film thickness, J & W Scientific) was used in the Automass (JEOL)-6890N (Agilent technologies) for GC-MS. A helium gas (1 ml/min) was used as a carrier gas. The injection temperature was 250° C. After injection, the column oven temperature was maintained at 80° C. for 1 minute, raised to 200° C. at a rate of 300° C./min, then to 250° C. at a rate of 5° C./min, then to 300° C. at a rate of 30° C./min, and maintained at 300° C. for 1 minute. The results of the analysis by GC-MS are shown in Table 1.

TABLE 1
GC-MS analysis data of products of metabolization by CYP714A2 protein

| | Metabolite | | |
|---|---|---|---|
| | and sample | Column | |
| | for | retention | |
| Substrate | comparison* | time (KRI) | Ion, m/z (relative intensity) |
| ent-kaurenoic acid | C13- | 2473 | 404 [M⁺] (9), 389(3), 348(3), 214(8), |
| | hydroxylated | | 193(100), 180(7), 73(58) |
| | (steviol) | | |
| | Steviol, | 2473 | 404 [M⁺] (9), 389(3), 348(3), 214(8), |
| | sample | | 193(100), 180(6), 73(48) |
| ent-7β- | C13- | 2594 | 492 [M⁺] (59), 477(6), 402(8), 343(3), |
| hydroxykaurenoic | hydroxylated^{#} | | 281(41), 208(17), 195(24), 193(25), |
| acid | | | 167(21), 73(100) |
| GA₁₂-7-aldehyde | C12ξ- | 2574 | 418 [M⁺] (3), 386(3), 296(7), 239(10), |
| | hydroxylated^{#} | | 211(100), 179(32), 151(78), 107(31), |
| | | | 73(50) |
| | C13- | 2547 | 418 [M⁺] (9), 403(5), 390(26), 261(8), |
| | hydroxylated^{#} | | 235(22), 208(63), 207(68), 193(100), |
| | | | 73(51) |
| GA₁₂ | C12α- | 2560 | 448 [M⁺] (2), 416(24), 388(16), 298(34), |
| | hydroxylated | | 239(55), 209(65), 207(76), 181(45), |
| | | | 180(47), 73(100) |
| | C12α- | 2560 | 448 [M⁺] (2), 416(29), 388(19), 298(45), |
| | hydroxylated, | | 239(65), 209(69), 207(78), 181(50), |
| | sample | | 180(49), 73(100) |
| | C12β- | 2580 | 448 [M⁺] (3), 416(30), 388(20), 298(42), |
| | hydroxylated, | | 239(51), 209(68), 207(78), 181(46), |
| | sample | | 180(46), 73(100) |
| | C13 | 2517 | 448 [M⁺] (26), 416(6), 389(10), 251(17), |
| | hydroxylated | | 235(16), 208(81), 207(100), 193(24), |
| | (GA₅₃) | | 181(60), 73(64) |
| | GA₅₃, sample | 2518 | 448 [M⁺] (21), 416(6), 389(8), 251(17), |
| | | | 235(16), 208(78), 207(100), 193(24), |
| | | | 181(62), 73(72) |
| | C12α, C13- | 2643 | 536 [M⁺] (23), 504(7), 477(9), 433(25), |
| | hydroxylated | | 420(14), 251(17), 193(62), 181(55), |
| | | | 147(30), 73(100) |
| | C12α, C13- | 2643 | 536 [M⁺] (22), 504(7), 477(10), 433(24), |
| | hydroxylated, | | 420(14), 251(16), 193(65), 181(56), |
| | sample | | 147(31), 73(100) |

*Me-TMSi derivative,
^{#}Refer to Gaskin and Macmillan (1991) GC-MS of the gibberellins and related compounds: Methodology and library of spectra

As shown in Table 1, steviol was identified as a metabolite of ent-kaurenoic acid. ent-7β,13-Dihydroxykaurenoic acid, in which a hydroxyl group was introduced to the 13th carbon, was similarly identified as a metabolite of ent-7β-hydroxykaurenoic acid. As metabolites of GA₁₂-7-aldehyde and GA₁₂, only a small amount of metabolites in which a hydroxyl group was introduced at the C-13 position were detected, and metabolites in which a hydroxyl group was introduced at the C-12α position were identified as major products. Therefore, CYP714A2, a cytochrome P450 enzyme of Arabidopsis thaliana, is an enzyme which introduces a hydroxyl group at the C-13 position of ent-kaurenoic acid with the B-ring having a six-membered ring ent-kaurane skeleton and ent-7β-hydroxykaurenoic acid but introduces a hydroxyl group at the C-12α position of GA₁₂-7-aldehyde with the B-ring having a 5-membered ring ent-gibberellane skeleton and GA₁₂ (FIG. 1).

### Example 2

### Preparation of Steviol Synthetase Gene Overexpressing Plant

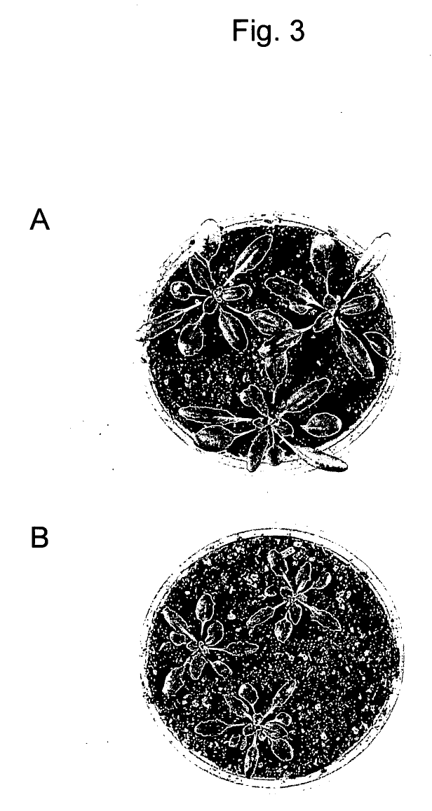

PCR was performed using a cDNA clone of the steviol synthetase gene prepared in Example 1 as a template, PCR primers At5g24900F (BamHI) (CCGGATCCATGGAGAGTTTGGTTGT [SEQ ID NO: 5]: the BamHI restriction enzyme site was positioned immediately before the translation start codon [underlined]) and At5g24900R (PstI) (CCCTGCAGTCAAACAACCCTAATGA [SEQ ID NO: 6]: the PstI restriction enzyme site was positioned immediately after the stop codon [underlined]) to introduce the restriction enzyme sites. The product obtained by PCR was cloned into a plasmid vector by ligation, and the nucleotide sequence was confirmed. A cDNA fragment of the steviol synthetase gene obtained by digesting this plasmid with BamHI and PstI was ligated to the BamBI/PstI site between the cauliflower mosaic virus 35S promoter (potent constitutive expression promoter) and the NOS terminator in the pCGN binary vector. This binary vector was introduced into Agrobacterium EHA105 by electroporation. Arabidopsis thaliana (ecotype Col-0) was transformed by the Floral-dip method. The obtained Ti seeds were aseptically seeded in a 1/2 Murashige-Skoog (MS) agar medium containing 50 mg/l of kanamycin and screened for transformants showing kanamycin resistance. T3 individuals, the posterity thereof, that homozygously have the introduced gene were used in experiments. As shown in FIG. 3, the obtained steviol synthetase gene overexpressing plant showed a phenotype of semidwarfism. In FIG. 3, photograph A shows wild-type Arabidopsis thaliana, and photograph B shows steviol synthetase gene overexpressing Arabidopsis thaliana.

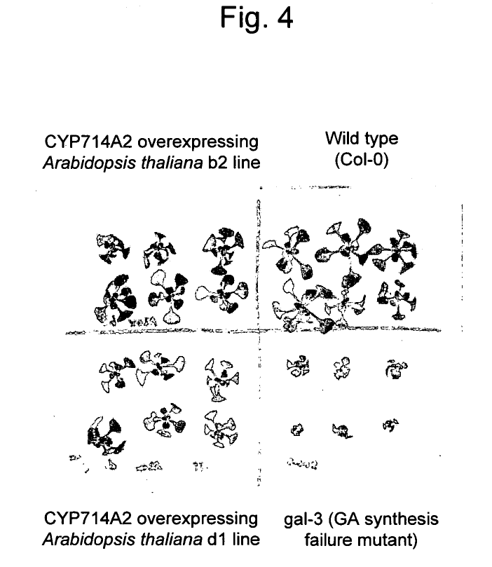

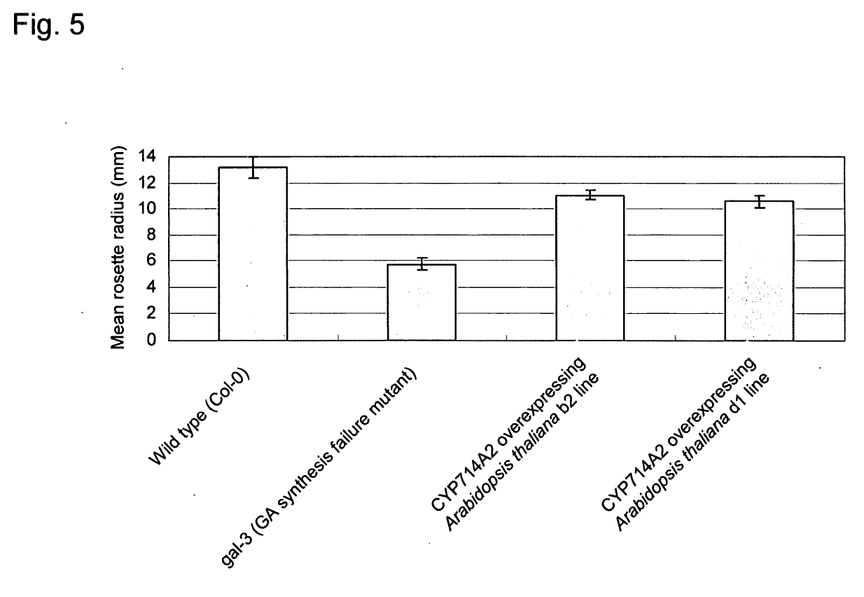

Growth measuring test was performed using two independent lines of the steviol synthetase gene overexpressing Arabidopsis thaliana prepared in this example (designated as b2 and d1), wild-type (Col-0), and gibberellin (GA) synthesis failure mutant. As the GA synthesis failure mutant, a mutant designated as gal-3 was used. In this test, shoot plants were transplanted in identical agar media at 6 days after seeding, and the rosette radius was measured at 10 days. Photographs of b2, d1, Col-1, and gal-3 at 10 days after transplantation are shown in FIG. 4. In this test, the rosette radius for each was obtained as a mean of 6 individuals. The measurement results are shown in Table 2 and FIG. 5.

TABLE 2

| | Col-0 | gal-3 | 35S::CYP714A2_b2 | 35S::CYP714A2_d1 |
|---|---|---|---|---|
| 1 | 15 | 7.5 | 10.5 | 11 |
| 2 | 12.5 | 6 | 11 | 11 |
| 3 | 15.5 | 6.5 | 12 | 9 |
| 4 | 11 | 4.5 | 12 | 10 |
| 5 | 14 | 4.5 | 11.5 | 12.5 |
| 6 | 11 | 5.5 | 9.5 | 10 |
| mean | 13.17 | 5.75 | 11.08 | 10.58 |
| SEM | 0.80 | 0.48 | 0.40 | 0.49 |

As shown in Table 2 and FIG. 5, the steviol synthetase gene overexpressing Arabidopsis thaliana prepared in this example significantly showed dwarfism as compared with the wild strain, and significantly increased the size as compared with the GA synthesis failure mutant. Thus, it was revealed that the steviol synthetase gene overexpressing Arabidopsis thaliana prepared in this example show very characteristic semidwarfism.

### Example 3

### Quantification of ent-kaurenoic Acid and Steviol in Steviol Synthetase Gene Overexpressing Arabidopsis thaliana

Two independent lines of the steviol synthetase gene overexpressing Arabidopsis thaliana prepared in Example 2 (designated as b2 and d1) and the wild-type Arabidopsis thaliana (Col-0) were grown under white light for 24 hours. 40 ng of 17,17⁻²H₂-labeled gibberellin was added to an aerial part immediately before bolting having a fresh weight of 5 g as an internal standard and extracted with 80% acetone. The extract was dried, then partitioned into solvents with 50% acetonitrile and n-hexane, and dried. The following two elution fractions were prepared.

Elution fraction 1: The hexane partition (containing kaurenoic acid) was subjected to silica gel chromatography. The sample was suspended in hexane and loaded on a silica gel column. After elution with hexane, the kaurenoic acid fraction was eluted with hexane:ethyl:acetate (85:15).

Elution fraction 2: The 50% acetonitrile partition (containing steviol) was suspended in 1% formic acid and loaded on the Oasis HLB column (Waters Corporation). After elution with 1% formic acid/40% acetonitrile, the steviol fraction was eluted with 1% formic acid/80% acetonitrile.

The elution fractions 1 and 2 were dissolved in methanol and loaded on the Bond Elut DEA column (Varian, Inc.). After elution with 100% methanol, the kaurenoic acid and steviol fractions were eluted with 0.1% acetic acid/methanol.

The obtained 0.1% acetic acid/methanol partition was fractionated by ODS-HPLC. In ODS-HPLC, SHISEIDO MGII5 (4.6 mm I.D.×250 mm) was used as the column. The column thermostat was maintained at 40° C. The mobile phase was 50% MeOH (1% AcOH) from 0 min to 5 min, with a gradient to obtain 100% MeOH at 25 min, and the partition was eluted with 100% MeOH until 35 min. The flow rate was 1 ml/min. The HPLC fraction was collected at 1 min/tube, and fractions 25 and 26 (containing steviol) and fractions 29, 30, and 31 (containing kaurenoic acid) were obtained.

Each fraction was collected, dried by concentration, derivatized with MSTFA, and analyzed by GC-MS. The DB-1 column (0.25 mm×15 m, 0.25 μm film thickness, J & W Scientific) was used in the Automass (JEOL)-6890N (Agilent technologies) for GC-MS. At this time, a helium gas (1 ml/min) was used as a carrier gas. The injection temperature was 250° C. The column oven temperature was maintained at 80° C. for 1 minute after injection, raised to 200° C. at a rate of 30° C./min, then raised to 280° C. at a rate of 5° C./min, and then maintained at 280° C. for 1 minute.

The results of quantification of ent-kaurenoic acid and steviol in the wild-type Arabidopsis thaliana and the steviol synthetase gene overexpressing Arabidopsis thaliana are shown in Table 3 (In Table 3, the unit is “pg/g fresh weight”).

TABLE 3

| | Wild-type | Steviol synthetase gene overexpressing | |
|---|---|---|---|
| | Arabidopsis | Arabidopsis thaliana | |
| | thaliana | Line b2 | Line d1 |
| ent-Kaurenoic | 1090 | 170 | 50 |
| acid | | | |
| Steviol | 10 | 170 | 150 |

As shown in Table 3, ent-kaurenoic acid, the substrate of steviol synthetase, in steviol synthetase gene overexpressing Arabidopsis thaliana was decreased to 5% to 16% of the wild-type. On the other hand, steviol, the metabolite of steviol synthetase increased to 15 to 17 times that in the wild-type.

### Example 4

### Quantification of Active Gibberellins (GA₄, GA₁) in Steviol Synthetase Gene Overexpressing Arabidopsis thaliana

17,17⁻²H₂-labeled gibberellin was added to overexpressing Arabidopsis thaliana (b2 and d1) used in Example 3 and the wild-type (Col-0) plant body and extracted with 80% acetone. The extract was dried and partitioned into solvents with 50% acetonitrile and n-hexane. The 50% acetonitrile partition was dried, then suspended in 500 mM phosphoric acid buffer (pH 8.0), and loaded on a polyvinylpyrrolidone column (Tokyo Chemical Industry Co., Ltd.). The resultant was eluted with 100 mM phosphoric acid buffer (pH 8.0), adjusted to pH 3.0 with hydrochloric acid, and then loaded on the Oasis HLB column (Waters Corporation). The resultant was eluted with 2% formic acid, and the gibberellin fraction was eluted with 1% formic acid/80% acetonitrile. The resultant was dried, dissolved in methanol, and loaded on the Bond Elut DEA column (Varian, Inc.). The resultant was eluted with 100% methanol, and the gibberellin fraction was eluted with 0.5% acetic acid/methanol. The resultant was dried, suspended in chloroform/ethyl acetate (1:1) containing 1% acetic acid, and allowed to pass through the SepPak silica cartridge (Varian, Inc.). The resultant solution was dried, and the residue was dissolved in water to perform analysis by LC-MS/MS. LC-MS/MS was performed using a quadruple/time-of-flight tandem mass spectrometer (Q-T of Premier, Waters Corporation) and Acquity Ultra Performance LC (Waters Corporation) and the Acquity UPLC BEH-C18 column (2.1×50 mm, 1.7 μm particle size, Waters Corporation). After elution with 98% acetonitrile (containing 0.05% acetic acid) over 5 minutes, the resultant was eluted with a gradient from 3% to 65% acetonitrile over 20 minutes. The flow rate was 200 μL/min.

The results of quantification of gibberellin A₄ (GA₄) and gibberellin A₁ (GA₁) in wild-type Arabidopsis thaliana and steviol synthetase gene overexpressing Arabidopsis thaliana are shown in Table 4 (the unit is “pg/g fresh weight” in Table 4).

TABLE 4

| | |
|---|---|
| Wild-type | Steviol synthetase gene overexpressing |
| Arabidopsis | Arabidopsis thaliana |

| | thaliana | Line b2 | Line d1 |
|---|---|---|---|
| GA₄ | 274 | Below detection | Below detection |
| | | limit | limit |
| GA₁ | 69 | 9799 | 8901 |

As shown in Table 4, GA₄, active gibberellin not having a hydroxyl group at the C-13 position, decreased to the below detection limit in steviol synthetase gene overexpressing Arabidopsis thaliana. On the other hand, GA₁ in active gibberellin having a hydroxyl group at the C-13 position increased to 129 to 142 times that of the wild-type Arabidopsis thaliana.

1. A steviol synthetase gene comprising any of the following polynucleotides (a) to (c):
(a) a polynucleotide encoding a protein which comprises the amino acid sequence of SEQ ID NO: 2;
(b) a polynucleotide encoding a protein which comprises an amino acid sequence of SEQ ID NO: 2 with deletion, substitution, addition, or insertion of one or more amino acids and has a function of hydroxylating the 13th carbon of ent-kaurenoic acid; and
(c) a polynucleotide encoding a protein which comprises an amino acid sequence having homology of 70% or more with the amino acid sequence of SEQ ID NO: 2 and has a function of hydroxylating the 13th carbon of ent-kaurenoic acid.

2. The steviol synthetase gene according to claim 1, which is a polynucleotide of the above-mentioned (b) or (c) and is derived from a plant selected from the group consisting of rice plant, Populus nigra, Rubus suavissimus, and stevia.

3. An expression vector comprising the steviol synthetase gene according to claim 1.

4. A transformant cell into which the steviol synthetase gene according to claim 1 is functionally incorporated.

5. A transformant plant into which the steviol synthetase gene according to claim 1 is functionally incorporated.

6. A method for producing steviol comprising extraction of steviol from a transformant into which the steviol synthetase gene according to claim 1 is incorporated so as to be overexpressed.

7. The method for producing steviol according to claim 6, wherein ent-kaurenoic acid is added as a substrate of steviol synthetase encoded by the gene.

8. The method for producing steviol according to claim 6, wherein the transformant is a plant.

9. The method for producing steviol according to claim 6, wherein the transformant is a gibberellin producing fungus.

10. A method for changing a ratio of gibberellin A₁ and gibberellin A₄ in a target cell, wherein the steviol synthetase gene according to claim 1 is overexpressed.

11. An expression vector comprising the steviol synthetase gene according to claim 2.

12. A transformant cell into which the steviol synthetase gene according to claim 2 is functionally incorporated.

13. A transformant plant into which the steviol synthetase gene according to claim 2 is functionally incorporated.

14. A method for producing steviol comprising extraction of steviol from a transformant into which the steviol synthetase gene according to claim 2 is incorporated so as to be overexpressed.

15. A method for changing a ratio of gibberellin A₁ and gibberellin A₄ in a target cell, wherein the steviol synthetase gene according to claim 2 is overexpressed.


SEQUENCE LISTING (6 sequences)

SEQ ID NO: 1 - DNA, 1578 bp, Arabidopsis thaliana
atggagagtttggttgttcatacggtaaatgcaatttggtgcatagttattgtcggaatcttcagcgtaggttatcatgtgtatggaagagcggtggtggagcagtggaggatgcggaggagtttaaagttgcaaggcgtgaagggtcctccgccgtcgatctttaacggcaatgtgtcggagatgcaacggattcagtcggaggctaaacactgttccggcgataacatcatttctcatgactattcttcttctctatttcctcatttcgatcactggcgaaaacaatacggaaggatttacacatactcaacggggttaaagcagcacctttacataaaccacccggaaatggtgaaggagcttagccaaaccaacacacttaaccttggtagaatcactcacatcaccaaacgccttaaccccattctcggcaatggcatcatcacctctaatgggcctcattgggcccatcaacgtcgtatcattgcctatgagtttacccacgacaaaatcaagggaatggttggtttaatggtggaatctgccatgccaatgttgaacaaatgggaagagatggtgaaaagaggaggagaaatgggttgtgacataagagtggacgaagaccttaaggatgtctcagctgatgtcatcgctaaggcttgctttgggagctctttttcaaaaggcaaagcaatattctctatgattagggatcttttaaccgccattactaagcgaagcgtcctcttcagattcaatggcttcactgatatggtgtttggaagtaagaagcatggtgatgtggatattgatgcgcttgagatggaattagaatcttctatatgggaaacggttaaggagagggaaattgaatgtaaggatactcacaagaaggatctaatgcagttgatactcgagggagcgatgcgaagctgcgatggtaacttgtgggacaagtcagcctatagacggtttgtggtggacaattgcaagagcatctatttcgccggacatgattcaaccgcagtctcagtgtcttggtgccttatgctcctcgctctcaatcctagttggcaggttaaaattcgcgatgaaatcttgagttcttgcaagaatggcattcccgacgcagaatcaattcctaatctcaaaacggtgacaatggtaatacaagaaacaatgagactatacccaccagcaccaatcgtgggaagagaagcatccaaagacataagacttggagaccttgtggtgccaaaaggagtgtgcatttggacactcattcctgccttacaccgagaccccgagatctggggaccagacgcaaacgacttcaagccagagaggtttagtgagggaatctctaaggcttgcaaataccctcagtcatacatcccatttggccttggaccaagaacatgcgtaggcaaaaactttggtatgatggaagtgaaagtgcttgtttcacttattgtctcaaagttcagttttactctttccccgacttatcagcactctccaagccataaactccttgtagagcctcaacatggtgttgtcattagggttgtttga

SEQ ID NO: 2 - protein, 525 aa, Arabidopsis thaliana
MESLVVHTVNAIWCIVIVGIFSVGYHVYGRAVVEQWRMRRSLKLQGVKGPPPSIFNGNVSEMQRIQSEAKHCSGDNIISHDYSSSLFPHFDHWRKQYGRIYTYSTGLKQHLYINHPEMVKELSQTNTLNLGRITHITKRLNPILGNGIITSNGPHWAHQRRIIAYEFTHDKIKGMVGLMVESAMPMLNKWEEMVKRGGEMGCDIRVDEDLKDVSADVIAKACFGSSFSKGKAIFSMIRDLLTAITKRSVLFRFNGFTDMVFGSKKHGDVDIDALEMELESSIWETVKEREIECKDTHKKDLMQLILEGAMRSCDGNLWDKSAYRRFVVDNCKSIYFAGHDSTAVSVSWCLMLLALNPSWQVKIRDEILSSCKNGIPDAESIPNLKTVTMVIQETMRLYPPAPIVGREASKDIRLGDLVVPKGVCIWTLIPALHRDPEIWGPDANDFKPERFSEGISKACKYPQSYIPFGLGPRTCVGKNFGMMEVKVLVSLIVSKFSFTLSPTYQHSPSHKLLVEPQHGVVIRVV

SEQ ID NO: 3 - DNA, 31 bp, Artificial
  Synthetic DNA
cgggatccatggagagtttggttgttcatac

SEQ ID NO: 4 - DNA, 31 bp, Artificial
  Synthetic DNA
ggggtacctcaaacaaccctaatgacaacac

SEQ ID NO: 5 - DNA, 25 bp, Artificial
  Synthetic DNA
ccggatccatggagagtttggttgt

SEQ ID NO: 6 - DNA, 25 bp, Artificial
  Synthetic DNA
ccctgcagtcaaacaaccctaatga